(19) United States
(12) Patent Application Publication
(10) Pub. No.: US 2018/0125446 A1
(43) Pub. Date: May 10, 2018

(54) SYSTEM AND METHOD FOR PRECISION DIAGNOSIS AND THERAPY AUGMENTED BY CANCER GRADE MAPS

(71) Applicant: KONINKLIJKE PHILIPS N.V., EINDHOVEN (NL)

(72) Inventors: Lilla Boroczky, Mount Kisco, NY (US); Amir Mohammad TAHMASEBI MARAGHOOSH, Melrose, MA (US); Shyam Bharat, Arlington, MA (US)

(21) Appl. No.: 15/572,180

(22) PCT Filed: May 20, 2016
(86) PCT No.: PCT/EP2016/061461

§ 371 Date: Nov. 7, 2017

Related U.S. Application Data
(60) Provisional application No. 62/170,710, filed on Jun. 4, 2015.

(51) Int. Cl. A61B 8/08; A61B 10/02; A61B 8/12; A61B 8/00; G06T 7/00; G06K 9/62

(52) U.S. Cl. CPC A61B 8/08; A61B 10/0241; A61B 8/12; A61B 8/463; G06T 7/41; G06T 7/0012; G06K 9/6267; A61B 8/5223; A61B 8/485

(57) ABSTRACT

An ultrasound system for performing cancer grade mapping includes an ultrasound imaging device (10) that acquires ultrasound imaging data. An electronic data processing device (30) is programmed to generate an ultrasound image (34) from the ultrasound imaging data, and to generate a cancer grade map (42) by (i) extracting sets of local features from the ultrasound imaging data that represent map pixels of the cancer grade map and (ii) classifying the sets of local features using a cancer grading classifier (46) to generate cancer grades for the map pixels of the cancer grade map. A display component (20) displays the cancer grade map, for example overlaid on the ultrasound image as a color-coded cancer grade map overlay. The cancer grading classifier is learned from a training data set (64) comprising sets of local features extracted from ultrasound imaging data at biopsy locations and labeled with histopathology cancer grades.

### FIELD

The following relates generally to the oncology diagnosis and treatment arts, biopsy and tissue sample collection arts, image-guided medical procedure arts, and related arts. It is described with particular reference to prostate cancer diagnosis and treatment, but will find application in the diagnosis and treatment of other types of cancer such as liver cancer, breast cancer, or so forth.

### BACKGROUND

As of year 2014, prostate cancer is the most common type of cancer in men, and the second leading cancer-related cause of mortality, in the United States. Annually, over 230,000 American men are diagnosed with prostate cancer, and close to 30,000 die of the disease. Prostate cancer is suspected if there are increased levels of prostate-specific antigen (PSA) in the blood, a palpable nodule, family history of prostate cancer or hypoechoic regions are seen in ultrasound images of the prostate. However, blood PSA test results produce a high false positive rate, which can lead to unnecessary treatment procedures with the associated possible complications.

More definitive prostate cancer diagnosis is conventionally by way of histopathology analysis of a biopsy sample acquired using a rectal tool guided by transrectal ultrasound imaging. Unfortunately, prostate cancer tends to form as scattered malignant regions, so that the false negative rate for this test is high due to poor targeting. A “false negative” in this sense includes a complete miss (falsely indicating no cancer), or a lower cancer grade than the highest grade cancer that is actually present in the prostate. More particularly, transrectal ultrasound-guided biopsies typically have a low sensitivity, with positive predictive values ranging from 40% to 60% hindering effective treatment planning and targeting. Biopsies are expensive and invasive, with possible complications; hence, repeat biopsies are not desirable, apart from being inefficient from a workflow perspective.

After a diagnosis of prostate cancer is made, an appropriate therapy is developed. Focal therapies such as high-intensity focused ultrasound (HIFU), cryotherapy, radio frequency ablation (RFA), or photodynamic therapy (PDT) are generally minimally invasive techniques that are designed to target the scattered regions of prostate cancer while minimally affecting the prostate organ. However, the scattered nature of typical prostate cancer makes effective targeting of high grade cancer regions via focal therapy a challenging task.

The following discloses a new and improved systems and methods that address the above referenced issues, and others.

### SUMMARY

In one disclosed aspect, an ultrasound system comprises: an ultrasound imaging device configured to acquire ultrasound imaging data; an electronic data processing device programmed to generate a cancer grade map by (i) extracting sets of local features from the ultrasound imaging data that represent map pixels of the cancer grade map and (ii) classifying the sets of local features using a cancer grading classifier to generate cancer grades for the map pixels of the cancer grade map; and a display component configured to display the cancer grade map.

In another disclosed aspect, an ultrasound method comprises: acquiring ultrasound imaging data; generating an ultrasound image from the ultrasound imaging data; generating a cancer grade map from the ultrasound imaging data by applying a cancer grading classifier to sets of local features extracted from the ultrasound imaging data; and displaying at least one of (i) the cancer grade map and (ii) a fused image combining the ultrasound image and the cancer grade map.

In another disclosed aspect, a non-transitory storage medium stores instructions readable and executable by an electronic data processing device to perform a cancer grade mapping method comprising: extracting sets of local features representing map pixels of a cancer grade map from ultrasound imaging data; and classifying each set of local features using a cancer grading classifier to generate a cancer grade for the corresponding map pixel of the cancer grade map. The cancer grade map comprises said map pixels with map pixel values equal to the cancer grades generated for the respective map pixels.

One advantage resides in providing a cancer grade map acquired via ultrasound.

Another advantage resides in providing such a cancer grade map in real-time.

Another advantage resides in providing improved biopsy sample collection using such a cancer grade map.

Another advantage resides in providing improved cancer therapy targeting using such a cancer grade map.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a transrectal ultrasound system providing a cancer grade map as disclosed herein.

FIG. 2 diagrammatically illustrates an ultrasound imaging method suitably performed using the system of FIG. 1 including displaying the cancer grade map superimposed on a b-mode ultrasound image.

FIG. 3 diagrammatically illustrates offline processing, suitably performed by a computer or other electronic data processing device, to generate the cancer grading classifier(s) employed in the system of FIG. 1.

### DETAILED DESCRIPTION

Grading of prostate cancer is typically by histopathology using samples acquired by transrectal ultrasound-guided biopsy. However, the ultrasound typically indicates (at best) the location of suspicious regions of the prostate, but cannot determine the cancer grade of these regions (or even whether they are cancerous at all). Thus, there is no assurance that the biopsy will acquire samples of the highest grade cancer present in the prostate. Further, the transrectal nature of the procedure tends to limit the number of samples that can be practically collected. Repeated transrectal biopsy procedures are also undesirable.

It is disclosed herein to generate a cancer grade map from the transrectal ultrasound image, leveraging existing raw “RF” time series data acquired by the transrectal ultrasound imaging. (The term “RF” conventionally denotes “radio frequency”. In the context of ultrasound, the imaging ultrasonic pulses are at a sonic frequency that is typically in the megahertz range which is comparable to radio frequencies; hence the term “RF” time series in the ultrasound context.) In typical ultrasound imaging, ultrasonic pulses are applied on the order of 30-50 times per second, thus generating 30-50 brightness images (called “b-mode” images in 2D ultrasound imaging) per second. It is known that these images may vary over time due to various possible mechanisms such as tissue heating or acousto-mechanical effects, so that for each pixel of the b-mode image one can generate a corresponding time-varying signal from the RF time series. These time-varying signals have been shown to correlate with tissue type in some instances.

As disclosed herein, the pixel-level RF time series information is used to generate a cancer grade map that can be overlaid onto a 2D image (e.g. b-mode image) or 3D image (for 3D ultrasound systems). In view of the (presently) poorly understood physical mechanisms leading to tissue contrast in RF time series data, a machine learning approach is employed in the disclosed embodiments. To this end, local features such as texture or wavelets are extracted for each map pixel. These map pixels may be at the pixel resolution of the ultrasound image, or may be at a coarser mapping resolution. (Moreover, the term “pixel” as used herein denotes “picture element” and may be either a 2D pixel or a 3D pixel depending on whether the RF time series data are acquired using a 2D ultrasound or 3D ultrasound system.) The local features form a feature vector representing each map pixel, which is input to a cancer grading classifier to assign a cancer grade for the map pixel. The cancer grading classifier (or classifiers) is trained using machine learning on labeled training data comprising ultrasound images of actual biopsy locations for which histopathology grades have been assigned. The cancer grade map may be overlaid as a color overlay on the b-mode image or otherwise fused with the ultrasound image.

The cancer grade map generation is fast. The trained classifier is computationally efficient, and the training can be performed offline. The ultrasound cancer grade mapping also uses the “raw” RF time series data already generated during conventional (e.g. b-mode) ultrasound imaging. Hence, the disclosed cancer grade mapping is readily employed during real-time ultrasound imaging. The cancer grade map can thereby be updated in real-time to account for rectal probe repositioning, inadvertent patient movement, changes in ultrasound imaging settings (e.g. resolution, focal point), or so forth. In addition to being used during the transrectal ultrasound-guided biopsy procedure, the approach is contemplated for use during brachytherapy seed implantation, during acquisition of planning images for inverse modulated radiation therapy (IMRT), or so forth.

While RF time series data are disclosed as the ultrasound imaging mechanism for generating the cancer grade mapping data, more generally mapping data generated by other contrast mechanisms such as elastography (in which ultrasonic pulses at a lower frequency are applied to induce tissue vibration) may be used. Moreover, while the illustrative embodiments employ transrectal ultrasound imaging for prostate cancer diagnosis and treatment, the approach is readily employed for real-time grading of other types of cancer such as liver or breast cancer.

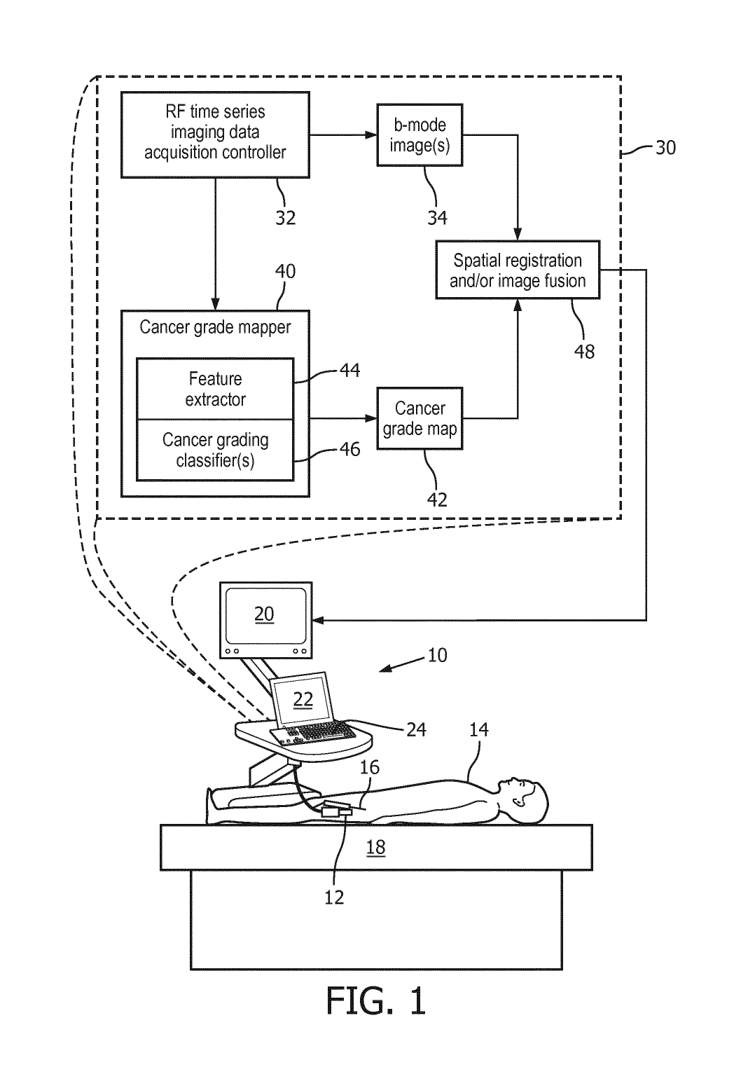

With reference to FIG. 1, a transrectal ultrasound system includes an ultrasound imaging system 10 (for example, an illustrated EPIQ™ ultrasound imaging system available from Koninklijke Philips N.V., Eindhoven, the Netherlands, or another commercial or custom-built ultrasound imaging system) with an rectal ultrasound probe 12 inserted into the rectum of a patient 14 and connected with the ultrasound imaging system 10 via cabling. (It will be appreciated that FIG. 1 is a diagrammatic representation; the ultrasound probe 12 is actually occluded from view when inserted into the patient's rectum). The illustrative ultrasound probe include an integrated biopsy needle 16 for collecting a biopsy sample; alternatively, a separate biopsy tool may be used, or the transrectal ultrasound system may be used for some other procedure, e.g. during IMRT planning image acquisition, which does not use a biopsy tool. For the transrectal ultrasound imaging procedure, the patient 14 lies on his side (as diagrammatically indicated in FIG. 1) on a diagrammatically indicated patient bed or support 18 with suitable pillows or other supports (not shown). The illustrative ultrasound imaging system 10 includes a display component 20 for displaying ultrasound images, and one or more user interfacing components such as a user interface display 22 and user input controls 24 (e.g. buttons, trackball, et cetera).

The ultrasound imaging system 10 further includes a microprocessor, microcontroller, or other electronic data processing component 30 which is diagrammatically indicated in FIG. 1, and which implements an RF time series imaging data acquisition controller 32 that is programmed to collect RF time series ultrasound imaging data and generate a conventional brightness (b-mode) image 34 from each frame of the RF time series ultrasound imaging data. In a typical setup, the controller 32 causes the ultrasound probe to inject sonic pulses (or pulse packets) at a chosen frequency (typically in the megahertz to tens of megahertz range, though frequencies outside this range, and/or multi-frequency pulses, are also contemplated) and acquire imaging data (known as a “frame”) in response to each such pulse or pulse packet. In this way, an RF time series of frames is acquired which typically includes 30-50 frames per second (other frame rates are contemplated). The data of each frame can be processed to form a two-dimensional image, e.g. a b-mode image, or in the case of a 3D ultrasound probe can be processed to form a 3D brightness image. The b-mode image is generated based on the echo delay (which correlates with depth) and direction (e.g. determined based on the phased array or beamforming settings of the ultrasound probe 12, or using a physical lens included with the probe). The b-mode image may, for example, be displayed on the display component 20, updated for every frame or every set of frames (e.g. averaging some chosen number of consecutive frames) so that the b-mode image is a real-time image.

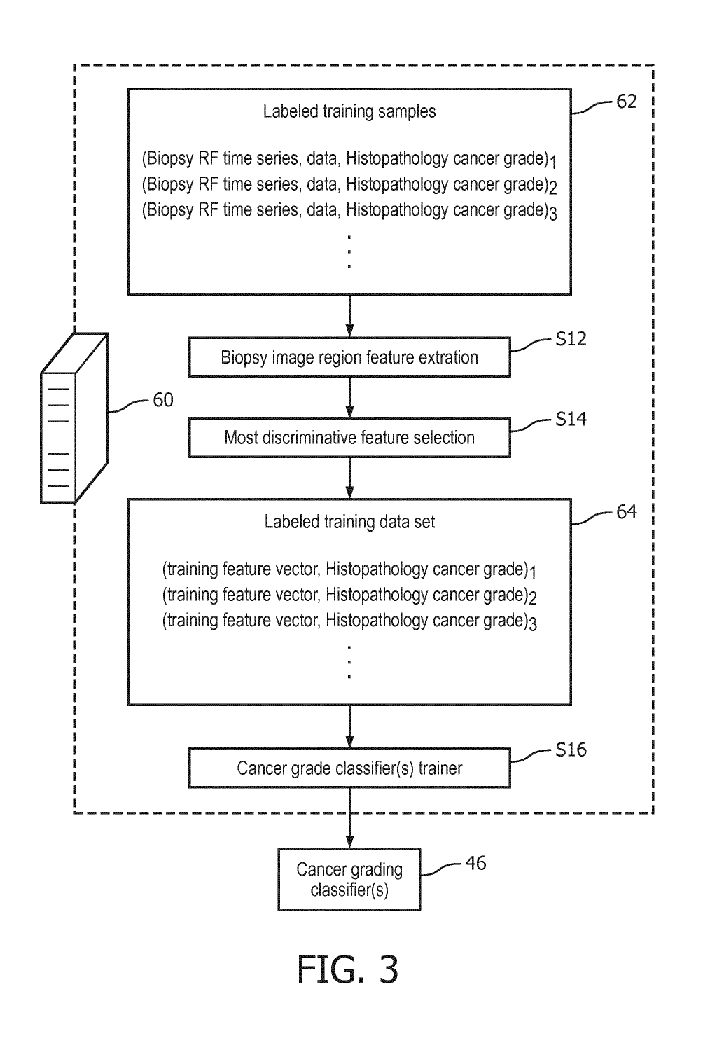

The RF time series ultrasound imaging data are also processed by a cancer grade mapper component 40, also implemented by suitable programming of the electronic data processing component 30 of the ultrasound imaging system 10, to generate a cancer grade map 42. The cancer grade map 42 is divided into an array of map pixels (which may be of the same resolution as the b-mode image 34, or of a coarser resolution, e.g. each map pixel may correspond to a contiguous n×n array of b-mode image pixels, e.g. a 3×3 array of b-mode image pixels, a 16×16 array of b-mode pixels, or so forth). For each map pixel, a feature extractor 44 of the cancer grade mapper 40 generates a feature vector representing the map pixel, and this feature vector is input to a cancer grading classifier (or set of cancer grading classifiers) 46 to generate a cancer grade for the map pixel. The cancer grade is preferably in accord with a standard cancer grading scheme, such as the Gleason score commonly used for histopathology grading of prostate cancers. The Gleason scoring system ranges from Grade 1 (normal prostate cells, i.e. benign), through Grades 2-4 in which an increasing fraction of the cells are irregular, to highest Grade 5 in which the cells are generally abnormal and randomly ordered. In a variant approach, two most common cell patterns are graded and the two scores are combined to generate a Gleason score between 2 and 10. The ultrasound imaging system 10 is incapable of imaging at the cellular level; however, the cancer grading classifier 46 was previously trained using training data comprising ultrasound image regions of biopsy sample locations paired with histopathology results for those biopsy samples (see FIG. 3 and related description herein) so that the output of the classifier 46 has a high correlation with the cancer grade that would be assigned by histopathological analysis of a sample taken from the location of the map pixel. In some embodiments, the classifier may employ a simplified or reduced grading scale: for example, the cancer grading classifier 46 may output values of 1, 3, or 5 where the value 3 spans Grades 2-4 of the Gleason scale.

This approach of ultrasound-based cancer grading is premised on the recognition that the increasing cell abnormality and increased randomness in cell ordering as cancer grade increases is likely to produce changes in ultrasound-induced tissue heating, and changes in acousto-mechanical response of the tissue. Since such phenomena are understood to produce time variation in the RF time series, the RF time series ultrasound data are reasonably expected to exhibit contrast for malignant tissue of different cancer grades. Similarly, in ultrasound elastography it is expected that malignant tissue of different cancer grades will exhibit different elasticity behavior due to changes at the cellular level and increased cellular disorder as the cancer grade increases, and hence ultrasound elastography is reasonably expected to exhibit contrast for malignant tissue of different cancer grades. The disclosed ultrasound cancer grading techniques leverage such cancer grade contrast to produce the cancer grade map 42 which provides cancer grading at about the resolution of the map pixel resolution.

The electronic data processing component 30 of the ultrasound imaging system 10 is further programmed to implement a spatial registration and/or image fusion component 48 which spatially registers (if necessary) the b-mode image 34 and the cancer grading map 42 in order to generate a fused image that is suitably displayed on the display component 20 of the ultrasound imaging system 10. Spatial registration may or may not be needed, depending upon the manner in which the b-mode image 34 is generated from the RF time series data—if this involves re-sizing, re-sampling, or so forth, then spatial registration may be needed. The image fusion can employ any suitable approach for combining the two images 34, 42. In one approach, the cancer grades (e.g. grades 1-5 of the Gleason scale) are assigned color codes, such as: Grade 1=transparent; Grade 2=yellow; Grade 3=yellowish orange; Grade 4=orange; and Grade 5=red (these are merely illustrative color codings). The color-coded cancer grading map are suitably fused with the b-mode image 34 as a semi-transparent overlay using, for example, alpha compositing (where the alpha value controlling the transparency of the cancer grading map overlay may optionally be a user-selectable parameter).

Some other contemplated image processing techniques for fusing the two images 34, 42 are as follows.

While image fusing is described in illustrative FIG. 1, other display presentation formats may be used, such as displaying the b-mode image 34 and the cancer grading map 42 side-by-side on the display component 20. The display may optionally include other features—for example, if the biopsy needle 16 includes a tracking feature that enables it to appear in the ultrasound image, its location may be indicated on the fused image. In such a case, an audible indicator could optionally be provided to indicate when the tracked biopsy needle tip enters a region of high-grade cancer as indicated by the cancer grading map 42 (e.g. the audible indicator could be a beeping sound whose frequency and/or loudness increases with increasing cancer grade penetrated by the needle; a flashing indicator light could be similarly activated). Moreover, while 2D ultrasound imaging is described, extension to 3D imaging is straightforward—in this case the displayed image may be a three-dimensional rendering, a projection image, or other image representation.

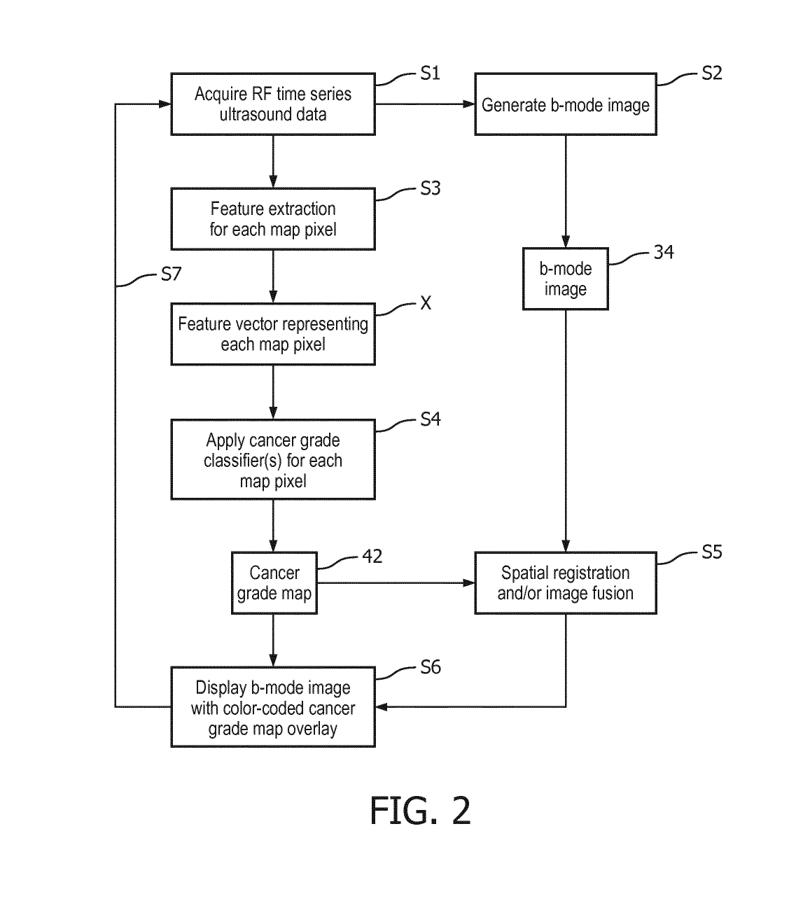

With continuing reference to FIG. 1 and with further reference to FIG. 2, a process suitably performed by the system of FIG. 1 is described. In an operation S1, the acquisition controller 32 operates the ultrasound imaging system 10 and probe 12 to acquire RF time series ultrasound data. These data are processed in an operation S2 to generate the b-mode image(s) 34. (Alternatively, another type of image representation may be generated.) In an operation S3, the feature extractor 44 is applied to extract a set (i.e. vector) of features for each map pixel. This processing entails the following: (1) generating a time series of values for each pixel of the image from the time series data; (2) concatenating contiguous n×n groups of image pixels to form the map pixels (unless n=1, i.e. the map pixels are of the same size as the image pixels); and (3) for each map pixel (that is, each n×n group of image pixels), extracting the set of features. The map pixel features should be local features, with each set of local features associated with an n×n group of image pixels forming a map pixel. Some suitable local features include, by way of illustration, texture features (such as standard textural features of Haralick et al., “Textural Features for Image Classification”, IEEE Transactions on Systems, Man, and Cybernetics vol. SMC-3, No. 6 pp. 610-621, 1973, or variants thereof) wavelet-based features, and/or spectral features. The output of the operation S3 is a feature set (i.e. feature vector) x representing (i.e. associated with) each map pixel. In an operation S4, the trained cancer grade classifier(s) 46 is (are) applied to the feature vector x of each map pixel to generate a cancer grade for the map pixel; these map pixel cancer grades then collectively define the cancer grade map 42. In an operation S5, the spatial registration/image fusion component 48 is applied to spatially register (if needed) the b-mode image 34 and the cancer grade map 42 and to fuse the two images 34, 42 to form the fused image, which is displayed on the display component 20 in an operation S6. The spatial registration, if needed, suitably entails aligning the images 34, 42 using rigid or elastic registration. For b-mode and RF modalities, the known processing and scan conversion steps from RF to b-mode can be used for the registration. The spatial registration can adjust the cancer grading map 42 to align with the b-mode image 34, or vice versa. It is also contemplated to perform the spatial registration to adjust the b-mode image 32 or the acquired RF time series data prior to performing the feature extraction and classification operations S3, S4 (that is, it is contemplated to spatially register the RF time series data and the b-mode image before generating the cancer grading map 42 from the RF time series data).

As indicated by a looping arrow S7 shown in FIG. 2, the processing may be iteratively repeated so as to update the b-mode image 34, the cancer grading map 42, and their fusion in real-time. The RF time series is acquired rapidly, e.g. 30-50 frames per second, making such real-time updating readily feasible. While illustrative FIG. 2 shows both the b-mode image 34 and the cancer grade map 42 being updated synchronously in each iteration of loop S7, this is not necessary. For example, the b-mode image 34 could be updated more frequently than the cancer grading map 42, e.g. the b-mode image could be updated every 10 frames while the cancer grade map 42 could be updated every 100 frames. A variant overlapping technique can be employed to facilitate updating the b-mode and cancer grade maps at the same rate. For example, if 100 RF time series frames are used to compute a grade map, the grade map display can start at b-mode image #101, using RF frames #1-#100. Then at b-mode image #102, the grade map calculated from RF frames #2-#101 is displayed, and so on. Thus, after an initial delay in starting the display of the cancer grade map 42 (to acquire the first 100 RF frames), the subsequent update of the cancer grade map 42 is at the same rate as the updating of the display of the b-mode image 34. (If the ultrasound probe 12 were moved, there would be a delay corresponding to acquisition of about 100 RF frames before the cancer grade map 42 is again synchronized; additionally, this overlapping technique is predicated on the grade map estimation being sufficiently fast).

With reference to FIG. 3, an illustrative method for employing machine learning to train the cancer grading classifier (or classifiers) 46 is described. This processing is optionally performed off-line, that is, by a computer 60 other than the microprocessor, microcontroller, or other electronic data processing component 30 of the ultrasound system 10. For example, the computer 60 may be a desktop computer, a notebook computer, a network-based server computer, a cloud computing system, or the like. The processing of FIG. 3 is performed before the patient procedure described with reference to FIG. 2, in order to provide the trained classifier 46.

The training of FIG. 3 operates on labeled training samples 62. Each labeled sample includes biopsy RF time series ultrasound data with locations of biopsy same extractions identified (for example on b-mode images generated from the RF time series data). Each biopsy location is labeled with its histopathology cancer grade, that is, the cancer grade assigned to the tissue sample extracted from the location by histopathological analysis of the tissue sample. The labeled training samples 62 are data for past patients who underwent transrectal ultrasound-guided prostate biopsy followed by histopathology grading of the samples, and for which the RF time series ultrasound data acquired during the biopsy were preserved. For each biopsy sample extraction of the training samples 62, the physician suitably labels the location on the b-mode image to provide a record of the location. The past patients whose data make up the training samples 62 are preferably chosen to provide a statistically representative sampling of positive samples: patients with prostate cancer in various stages as demonstrated by the histopathology results. The training samples 62 also preferably include a sampling of patients without prostate cancer (negative samples; these may also or alternatively be provided by patients with prostate cancer where the negative samples constitute biopsy samples drawn from areas of the prostate organ for which the histopathology indicated no cancer, i.e. Gleason score of one).

In an operation S12, the RF time series data are processed to generate a features set (i.e. feature vector) for map pixels encompassing each biopsy location. The operation S12 suitably corresponds to the operation S3 of FIG. 2, e.g. the same map pixel resolution and the same set of features, i.e. the same feature vector. In an alternative approach, the set of features is chosen as part of the machine learning training process of FIG. 3—in this case, the processing includes an optional operation S14 in which selects the local features that make up the feature vector extracted by the operation S3. Such feature selection can be performed manually or automatically, for example using mutual information, correlation, or similar statistics to identify and remove redundant features of an initial feature set to form the final feature set forming the feature vector used in operation S3. Other suitable feature selection algorithms include exhaustive search, a genetic algorithm, forward or backward elimination, or so forth.

In the case of local features extracted from RF time series ultrasound imaging data, the usual transrectal ultrasound imaging-guided biopsy procedure typically acquires the requisite RF time series ultrasound imaging data in due course as b-mode imaging is performed (because the b-mode image is generated from RF time series data). It will be appreciated that if, on the other hand, the operation S3 extracts features from some other type of ultrasound imaging data, such as elastography imaging data, then the ultrasound data of the labeled training samples 62 would need to include ultrasound data of the requisite type (e.g. elastography imaging data) in order to allow training sets of the corresponding local features to be extracted from the training ultrasound imaging data.

The output of operation S12 and optional operation S14 is a feature vector representing each map pixel corresponding to a biopsy location. (Depending upon the resolution with which the biopsy location is identified, there may be multiple map pixels spanning the biopsy location.) These feature vectors, each labeled with the histopathology cancer grade for the corresponding extracted tissue sample, form a labeled training set 64.

In an operation S16, the cancer grading classifier 46 is trained on this training set 64. The training optimizes parameters of the cancer grading classifier 46 so as to minimize the error between the outputs of the cancer grading classifier 46 for the input training feature vectors of the set 64 and their corresponding histopathology cancer grade labels. The cancer grading classifier 46 may comprise a single multi-label classifier, for example having discretized outputs 1-5 corresponding to the five Gleason scores. Alternatively, the cancer grading classifier 46 may comprise a set of binary classifiers, each for a different cancer grade—for example, the binary classifier for Gleason score 4 is trained to optimally output a “1” for those training feature vectors whose labels are Gleason score 4 and a “0” for those training vectors whose labels are otherwise. In some embodiments, the classifier 46 is an ensemble of classifiers, such as an ensemble of decision trees (sometimes called a random forest). Some suitable classifiers include, but are not limited to: linear regression, logistic regression, support vector machines, decision tree classifiers, and so forth. In case of the use of ensemble classifier the grade value of a map pixel can be derived such as the majority of the malignancy decision of each classifier.

Many such classifiers output continuous values. To generate discrete cancer grades, such as Gleason scores, a thresholding operation can be performed on the continuous-valued output of the classifier, so that the map pixel values are discrete values. Alternatively, no thresholding is performed and the map pixels are assigned the continuous-valued classifier outputs directly. In this case, the image fusion operation 48 may optionally perform color coding using a continuous spectrum of colors mapped to the continuous classifier output, rather than discretized colors as previously described.

The resulting trained cancer grading classifier 46 (or its trained parameters) are suitably loaded into the ultrasound system 10 for use by the microprocessor, microcontroller, or other electronic data processing component 30 in performing the cancer grade classification operation S4.

The system of FIG. 1 includes the real-time ultrasound imaging system 10, where, for example, the trans-rectal probe 12 is used to acquire images of the prostate organ. Images include but not limited to b-mode imaging, RF data, and elastography, or other RF data-based methods such as backscatter coefficient estimation, attenuation estimation, or so forth. The RF data provide additional information pertaining to cancer tissue with respect to conventional b-mode imaging. It will be recognized that some information is lost due to the various steps of signal processing entailed in transforming the raw RF time series data to b-mode images. As disclosed herein, using the ultrasound data (e.g. RF time series data, and/or elastography data, and/or so forth), an estimation of cancer grade is performed by using pattern recognition and machine learning techniques to estimate the grade of each map pixel or region in the prostate. The cancer grade for each voxel or region (i.e. map pixel) is computed, and the cancer grade map 42 is formed. The cancer grade map 42 can be overlaid on a b-mode image of the prostate, or can be rendered in 3D if the ultrasound device 10 acquires 3D ultrasound imaging data. The cancer grade map 42 can be used by the ultrasound imaging and biopsy system to better position the probe 12 or biopsy device 16. Once the ultrasound probe 12 is moved to a particular location, the ultrasound imaging system 10 acquires updated ultrasound images which are graded by the cancer grade mapper 40, so as to update the cancer grade values, and the cancer grade map 42 is thereby updated accordingly. This process can be repeated in real-time until a prostate region of high cancer grade as indicated by the cancer grade map 42 is identified. In the context of a biopsy application, the identified prostate region of high cancer grade is chosen as the biopsy target, and the biopsy gun or tool 16 is guided to this location to acquire a tissue sample from the high grade region.

A similar workflow is also contemplated for targeted therapy. In this application, the high grade cancer is identified, and chosen as a target for the therapy tool (e.g., needle delivering radioactive seeds in the case of brachytherapy, or a radiofrequency ablation needle, or so forth). In the case of brachytherapy, for example, a larger number of seeds may be placed at locations indicated in the cancer grade map 42 as being of high grade, and a lower number of seeds may be placed at locations indicated as lower grade. In an IMRT planning application, the cancer grade mapper 40 is employed during acquisition of planning images (for example, computed tomography, i.e. CT, planning images and alternately, with ultrasound RF time series to augment the planning CT data). The cancer grade map 42 is spatially registered with the planning images using fiduciary markers, anatomical markers, or so forth, and the aligned cancer grade map 42 provides sole or additional information for segmenting the high grade cancer region or regions in the planning image.

For purposes of grading cancer, the illustrative embodiment employs the cancer grade mapper 40 as a tool for guiding the biopsy procedure in order to perform targeted sampling of the regions of highest cancer grade as indicated by the ultrasound-generated cancer grade map 42. In this approach, the cancer grade map 42 serves to guide the biopsy sample collection, but the cancer grading produced by histopathology analysis of the biopsy samples serves as the accepted grading for clinical use (that is, for guiding diagnosis and treatment). This illustrative approach has the advantage that the clinical grading is histopathology grading which is well accepted by oncologists.

In an alternative embodiment, the ultrasound-generated cancer grade map 42 serves as the grading for clinical use. That is, in such embodiments no biopsy is performed, and instead the oncologist relies upon the cancer grade map 42 as the cancer grading. This approach requires that the specificity and sensitivity of cancer grading provided by the cancer grade map 42 satisfy clinical requirements, which can be determined over time by recording the grade that would be produced by the cancer grade map 42 and comparing it with the histopathology grade—if these exhibit satisfactory agreement over time and with sufficient statistics, then the cancer grade map 42 may be reasonably relied upon alone. This approach has the advantage of eliminating the invasive biopsy procedure as well as the delay between biopsy sample collection and the subsequent histopathology analysis and reporting.

The illustrative prostate cancer example employs the illustrative transrectal ultrasound probe 12 as such an approach is commonly and effectively used in ultrasound imaging of the prostate. However, as previously mentioned the disclosed ultrasound-based cancer grading approaches may be usefully employed to grade other types of cancer. Depending upon the type of cancer, a different type of ultrasound probe may be employed. For example, in breast cancer imaging a surface ultrasound probe may be preferable.

In the illustrative embodiments, the cancer grade mapper 40 is implemented by the microprocessor, microcontroller, or other electronic data processing component 30 which is a component of the ultrasound device 10. This is advantageous because the microprocessor or microcontroller 30 is integrated with the ultrasound device 10, for example also serving as its electronic controller in some embodiments, and accordingly has direct access to acquired ultrasound data including the raw RF time series data and can be integrated with image display functionality of the ultrasound device 10 in order to, for example, display the cancer grade map 42 as an overlay on the b-mode image. However, it is alternatively contemplated for the cancer grade mapper 40 to be implemented on a different electronic data processing device which receives the ultrasound imaging data including the RF time series data and includes a display component (or accesses the display component 20 of the ultrasound device 10) for displaying the cancer grade map 42. For example, the cancer grade mapper 40 may be implemented on a notebook computer connected with the ultrasound device 10 by a USB cable or other data connection. In such embodiments, the cancer grade mapper 40 may execute concurrently with the ultrasound imaging to update the cancer grade map 42 in real time as previously described; or, alternatively, the cancer grade mapper 40 may be executed after the ultrasound imaging session is completed, operating on saved RF time series ultrasound data.

It will be further appreciated that the various ultrasound-based cancer grading approaches such as those disclosed herein with reference to FIGS. 1 and 2 may be embodied by a non-transitory storage medium storing instructions that are readable and executable by the microprocessor, microcontroller, or other electronic data processing component 30 to perform these operations. Similarly, the various classifier training approaches such as those disclosed herein with reference to FIG. 3 may be embodied by a non-transitory storage medium storing instructions that are readable and executable by a computer or other electronic data processing component that performs the offline classifier training. Such non-transitory storage media may, by way of non-limiting illustration, include a hard disk drive or other magnetic storage medium, a flash memory, read-only memory (ROM) or other electronic storage medium, an optical disk or other optical storage medium, various combinations thereof, or so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An ultrasound system comprising:
an ultrasound imaging device configured to acquire ultrasound imaging data;
an electronic data processing device programmed to generate a cancer grade map by (i) extracting sets of local features from the ultrasound imaging data that represent map pixels of the cancer grade map and (ii) classifying the sets of local features using a cancer grading classifier to generate cancer grades for the map pixels of the cancer grade map; and
a display component configured to display the cancer grade map.

2. The ultrasound system of claim 1 wherein the electronic data processing device is programmed to extract the sets of local features representing map pixels from RF time series ultrasound imaging data.

3. The ultrasound system of claim 1 wherein:
the ultrasound imaging device is configured to acquire ultrasound imaging data including elastography imaging data in which ultrasonic pulses at a lower frequency are applied by the ultrasound device to induce tissue vibration; and
the electronic data processing device is programmed to extract the sets of local features representing map pixels from elastography imaging data.

4. The ultrasound system of a claim 1 wherein:
the electronic data processing device is further programmed to generate an ultrasound image from the ultrasound imaging data and to generate a cancer grade map overlay from the cancer grade map that is aligned with the ultrasound image; and
the display component is configured to display a fused image that combines the ultrasound image and the cancer grade map; and
wherein the electronic data processing device is programmed to generate the ultrasound image as a brightness (b-mode) image from ultrasound imaging data comprising RF time series ultrasound imaging data.

5. (canceled)

6. The ultrasound system of claim 4 wherein the electronic data processing device is programmed to generate the fused image as the ultrasound image overlaid with a color coded cancer grade map overlay in which cancer grades of the cancer grade map are represented by color coding; and
wherein the electronic data processing device is programmed to extract the sets of local features representing map pixels of the cancer grade map including one or more of (1) texture features, (2) wavelet-based features, and (3) spectral features.

7. The ultrasound system of claim 4 wherein the ultrasound system is configured to continuously acquire ultrasound imaging data and to update the ultrasound image, the cancer grade map, and the fused image in real-time using the continuously acquired ultrasound imaging data.

8. The ultrasound system of claim 1 wherein each map pixel of the cancer grade map consists of a contiguous n×n array of pixels of an ultrasound image generated from the acquired ultrasound imaging data, wherein n≥1.

9. (canceled)

10. (canceled)

11. The ultrasound system of claim 1 further comprising:
a rectal ultrasound probe) connected with the ultrasound imaging device wherein the ultrasound imaging device is configured to acquire ultrasound imaging data of a prostate organ using the rectal ultrasound probe, the electronic data processing device is programmed to generate a prostate cancer grade map by (i) extracting sets of local features from the ultrasound imaging data that represent map pixels of the prostate cancer grade map and (ii) classifying the sets of local features using a prostate cancer grading classifier to generate prostate cancer grades for the map pixels of the prostate cancer grade map, and the display component is configured to display the prostate cancer grade map.

12. The ultrasound system of claim 11 further comprising:
a rectal biopsy tool connected with the rectal ultrasound probe and configured to collect a prostate tissue biopsy sample;
wherein the electronic data processing device is further programmed to generate a prostate ultrasound image from the ultrasound imaging data and the display component is further configured to display a fused image combining the prostate ultrasound image and the prostate cancer grade map.

13. The ultrasound system of claim 1 further comprising:
an electronic data processing device programmed to generate the cancer grading classifier by machine learning on a labeled training data set comprising training sets of local features extracted from ultrasound imaging data at biopsy locations and labeled with histopathology cancer grades.

14. An ultrasound method comprising:
acquiring ultrasound imaging data;
generating an ultrasound image from the ultrasound imaging data;
generating a cancer grade map from the ultrasound imaging data by applying a cancer grading classifier to sets of local features extracted from the ultrasound imaging data; and
displaying at least one of (i) the cancer grade map and (ii) a fused image combining the ultrasound image and the cancer grade map.

15. The ultrasound method of claim 14 wherein:
the ultrasound imaging data includes RF time series ultrasound imaging data;
the ultrasound image comprises a brightness mode (b-mode) image generated from the RF time series ultrasound imaging data; and
the cancer grade map is generated from the RF time series ultrasound imaging data.

16. The ultrasound method of claim 14 wherein the displaying comprises displaying a fused image comprising the ultrasound image overlaid with a color-coded overlay representation of the cancer grade map.

17. The ultrasound method of claim 14 further comprising iteratively repeating the acquiring, the generating of the ultrasound image, the generating of the cancer grade map, and the displaying to update the displayed cancer grade map or fused image in real time.

18. (canceled)

19. (canceled)

20. The ultrasound method of claim 14 further comprising:
training the cancer grading classifier on a labeled training data set comprising training sets of local features extracted from ultrasound imaging data at biopsy locations and labeled with histopathology cancer grades.

21. (canceled)
